(19) United States
(12) Patent Application Publication
(10) Pub. No.: US 2024/0269042 A1
(43) Pub. Date: Aug. 15, 2024

(54) CLOSED STERILE MEDICATION MIXING AND/OR ADMINISTRATION SYSTEM WITH DISINFECTION OPTIONS

(71) Applicant: Hyprotek, Inc., Spokane, WA (US)

(72) Inventor: Patrick O. Tennican, Spokane, WA (US)

(21) Appl. No.: 18/439,826

(22) Filed: Feb. 13, 2024

Related U.S. Application Data
(60) Provisional application No. 63/445,958, filed on Feb. 15, 2023.

(51) Int. Cl. A61J 1/20; A61J 1/14

(52) U.S. Cl. CPC A61J 1/2096; A61J 1/1406; A61J 1/2065

(57) ABSTRACT

A mixing and/or administration system may include a syringe housing, a piston coupled to the syringe housing and including a valve (e.g., a push-push valve), an extension housing coupled to the piston, a vial positioned within or coupled to an open end of the extension housing, and a flexible packaging surrounding at least a portion of the mixing and/or administration system. In examples, the packaging may be sealed to one or more portions of the mixing and/or administration system such as the syringe housing, the piston, the push-push valve, the extension housing, and/or the vial. In examples, the mixing and/or administration system may include a separate pouch coupled to the packaging containing at least one cap or cleaning device. A tear strip may be included to open the separate pouch.

### RELATED APPLICATIONS

This application claims the benefit of priority to U.S. Provisional Application No. 63/445,958 filed on Feb. 15, 2023, which is incorporated herein by reference in its entirety.

### BACKGROUND

Preparation of medication agents and administration of such agents to an individual often involves mixing of two or more components to form the agent and subsequent delivery of the mixed medicant to the individual. For example, a practitioner (e.g., nurse, physician, caregiver, medical assistant, emergency personnel, etc.) may prepare and administer a medication to a patient. Alternatively, in some examples, a patient may prepare and administer a medication to themselves without the assistance of a practitioner. The mixing of components can typically involve extraction of one component in fluid form from a vial or other container and transfer of such components into a separate container which holds another component. A variety of problems may occur when utilizing conventional methodology and devices for mixing and/or administering medicants to an individual. For example, where multiple components are to be mixed, the extraction, introduction, and transfer of one component into another component can potentially expose one or both of the components to a non-sterile or contaminated environment leading to contamination. Additionally, one or both of the components to be mixed may be toxic or otherwise harmful and could expose the patient and/or a practitioner administering the medication to harm. Furthermore, the components to be mixed must be compatible in types and amounts and errors in matching can have deleterious consequences. To avoid these and other problems that frequently arise in the preparation and administration of medications, it would be desirable to develop effective systems and packaging for mixing and administering medication agents.

### BRIEF DESCRIPTION OF THE DRAWINGS

The detailed description is described with reference to the accompanying figures. In the figures, the left-most digit(s) of a reference number identifies the figure in which the reference number first appears. The use of the same reference numbers in different figures indicates similar or identical components or features.

FIG. 1A is a top view and partial cross-sectional view of a mixing and/or administration system in accordance with an example of the present disclosure.

FIG. 1B is a side view and cross-sectional view of the mixing and/or administration system in accordance with an example of the present disclosure.

FIG. 2A is a cross-sectional view of a push-push valve in a closed position in accordance with an example of the present disclosure.

FIG. 2B is a cross-sectional view of the push-push valve in an open position in accordance with an example of the present disclosure.

FIG. 3A is a perspective view of a mixing and/or administration system including packaging sealed to a portion of a piston in accordance with an example of the present disclosure.

FIG. 3B is a perspective view of the mixing and/or administration system in FIG. 3A including a second packaging sealed to the portion of the piston in accordance with an example of the present disclosure.

FIG. 3C is a perspective view of a mixing and/or administration system including packaging sealed to a syringe housing in accordance with an example of the present disclosure.

FIG. 3D is a perspective view of the mixing and/or administration system in FIG. 3C including a second packaging sealed to a syringe housing in accordance with an example of the present disclosure.

FIG. 3E is a perspective view of a mixing and/or administration system including packaging sealed to a connector in accordance with an example of the present disclosure.

FIG. 3F is a perspective view of the mixing and/or administration system in FIG. 3E including a second packaging sealed to a connector in accordance with an example of the present disclosure.

FIG. 3G is a perspective view of a mixing and/or administration system including packaging sealed to a Luer connector in accordance with an example of the present disclosure.

FIG. 3H is a perspective view of the mixing and/or administration system in FIG. 3G including a second packaging sealed to a Luer connector in accordance with an example of the present disclosure.

FIG. 4A is a perspective view of an alternative mixing and/or administration system including packaging sealed to a syringe housing in accordance with an example of the present disclosure.

FIG. 4B is a perspective view of the alternative mixing and/or administration system in FIG. 4B including a second packaging sealed to the syringe housing in accordance with an example of the present disclosure.

FIG. 4C is a perspective view of an alternative mixing and/or administration system including a port sealed to the packaging in accordance with an example of the present disclosure.

FIG. 4D is a perspective view of the alternative mixing and/or administration system in FIG. 4C including a separate packaging surrounding the port in accordance with an example of the present disclosure.

FIG. 4E is a perspective view of an alternative mixing and/or administration system including packaging sealed to a portion of the syringe housing in accordance with an example of the present disclosure.

FIG. 4F is a perspective view of the alternative mixing and/or administration system in FIG. 4E including a second packaging sealed to the portion of the syringe housing in accordance with an example of the present disclosure.

FIG. 4G is a perspective view of another alternative mixing and/or administration system 400G including a syringe in a first packaging, an IV bag in a second packaging, and IV tubing in a third packaging, in accordance with an example of the present disclosure.

FIG. 4H is a perspective view of another alternative mixing and/or administration system 400H including a syringe in a first packaging, an IV bag in a second packaging, and IV tubing in a third packaging, in accordance with an example of the present disclosure.

### OVERVIEW

This application describes a mixing and/or administration system (or assembly system) for combining and mixing components to produce an administration-ready agent such as a medicant (e.g., chemotherapy medication, vaccine, insulin, and the like). The mixing and/or administration system may be described as being a closed-system in that separate components of an agent can be combined and mixed without exposing the components to an environment external to the mixing and/or administration system and without exposing the patient or the practitioner that is administering the medicant to any airborne particles or vapor associated with the medicant. The mixing and/or administration system can include a syringe having a syringe housing configured to hold a mixing component. The syringe housing may include a first side and a second side opposite the first side. The first side of the syringe housing may include a connector (e.g., a Luer-lock connector) configured to receive a needle, alternative cannula, tubing and/or adapters that may be utilized during administering of the medicant within the syringe housing. In examples, the first end of the syringe housing may have a cap providing a fluid seal.

In examples, the second side of the syringe housing may be coupled to a piston having a fluid passageway (or channel) passing entirely through the length of the piston. The piston may have a first end and a second end opposite the first end. In examples, the first end of the piston may be coupled to the second end of the syringe housing. In examples, a valve may be associated with the piston and can be configured to allow selective fluid passage through the piston passageway. In examples, the valve may be a push-push valve, a push-pull valve, or a twist valve. In examples, the valve may be positioned anywhere along the length of the piston. In examples, the valve may be positioned or located proximate the second end of the piston. In examples, the piston may include a raised ridge proximate the second end of the piston, where the raised ridge is configured to prevent the syringe housing from abutting the valve when the piston is inserted into the syringe housing.

The mixing and/or administration system may include an extension housing coupled to a second end of the piston. The extension housing may have an open end (or receiving end) configured to receive a vial. In examples, the second end of the piston may include a puncture device configured to puncture a portion of the vial (e.g., a cap associated with the vial).

The mixing and/or administration system may include a packaging surrounding the mixing and/or administration system, in whole or in part. The packaging may be sealed around the enclosed mixing and/or administration system so that the mixing and/or administration system is maintained in a sterile environment. In examples, the packaging may be formed from a transparent, semi-transparent, or translucent material. In examples, a portion of the packaging may be transparent. For example, a first side of the packaging may be transparent and a second side opposite the first side may be opaque.

In examples, the packaging may be coupled to or bonded to one or more portions of the mixing and/or administration system in order to prevent the mixing and/or administration system from moving within the packaging. In examples, the packaging may be coupled to a portion of the syringe housing (e.g., the syringe flanges located on the syringe housing). In examples, the packaging may be sealed to or coupled to a portion of the piston. In examples, the packaging may be sealed to or coupled to a portion of the push-push valve. Sealing the packaging to a portion of the mixing and/or administration system (e.g., the push-push valve) makes it easier for an individual to manipulate the valve from outside the packaging (i.e., without opening the mixing and/or administration system) and prevents the syringe from sliding around inside the packaging.

In examples, the packaging may include multiple compartments or sections separated from one another by a hermetic seal. For instance, the packaging may include a first section housing a vial which is hermetically sealed from a second section housing one or more caps or cleaning devices. Cleaning devices may include one or more sponge applicators, alcohol wipes, and/or swabs, which may or may not carry an antimicrobial composition such as any of those described herein. The first section may remain sealed and sterile during use, while the second section may be opened to access the one or more caps or cleaning devices for use. In some examples, the packaging may include one or more tear strips to open a portion of the packaging. In examples, a tear strip may extend across an entire length or width of the packaging (e.g., from a first side to a second side of the packaging). In examples, the tear strip may partially extend across less than all of a length or width of the packaging. In examples, the tear strip may be positioned proximate the push-push valve located on the piston. In examples, the tear strip may be positioned proximate a first end of the syringe housing (e.g., proximate the end of the syringe housing that includes one or more caps or cleaning devices). In examples, the tear strip may be positioned proximate a second end of the syringe housing (e.g., proximate the end of the syringe housing that is coupled to the piston). In examples, the tear strip may be positioned proximate the syringe flanges. The syringe flanges may extend out from a side of the syringe housing and provide an area or surface for a user's fingers to grasp during aspiration or administration. In some examples, the tear strip may comprise a pull strip made of material which is stronger than a material from which the rest of the packaging is made. In some examples, guide tape may be applied adjacent the pull strip to guide or control tearing of the packaging material to produce a smooth, straight tear. In some examples, the packaging may be thinned, scored, or otherwise weakened in order to provide a localized frangible region or line along which the packaging is designed to tear during opening. However, regardless, of the construction of the tear strip, the packaging should provide a sterile sealed package until the tear strip is used to open a portion or section of the package.

An alternative mixing and/or administration system may include a syringe housing having a first end and a second end opposite the first end. A piston may be coupled to the first end of the syringe housing and a Luer-lock fitting may be coupled to the second end of the syringe housing. In examples, an adapter including a push-push valve may be coupled to the Luer-lock fitting. In examples, the adapter may include a port extending in a direction perpendicular or oblique to the push-push valve. In examples, an extension housing may be coupled to the adapter. The extension housing may include an open end (or receiving end) configured to receive a vial. In examples, a transparent or semi-transparent packaging may surround the entire alternative mixing and/or administration system or a portion of the alternative mixing and/or administration system. In examples, a tear-open pouch may be sealed or coupled to a portion of the packaging surrounding the mixing and/or administration system.

Although the figures depict the mixing and/or administration system as comprising syringes and the description presents methodology primarily in terms of preparing a medicant, it is to be understood that the disclosure encompasses alternative container shapes and adaptation of devices for use in mixing of components to form mixtures or agents other than medicants. For example, the mixing and/or administration system may be used to mix various epoxies, adhesives, sealants, paints, resins, etc. for use in various other industries such as dentistry, manufacturing, electronics, automotive repair, etc.

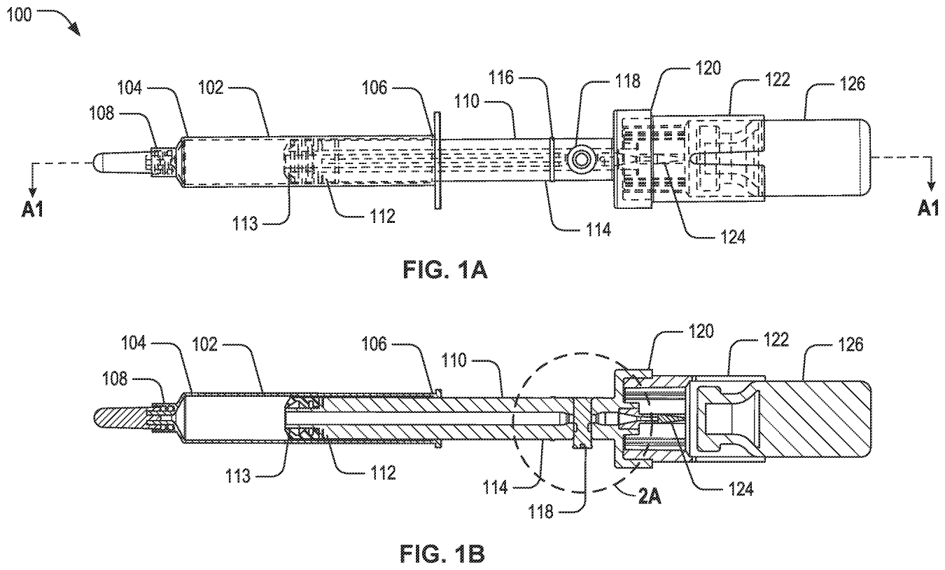

FIG. 1A is a top view and partial cross-sectional view of a mixing and/or administration system in accordance with an example of the present disclosure. FIG. 1B is a side view and cross-sectional view of the mixing and/or administration system 100 in accordance with an example of the present disclosure. The mixing and/or administration system 100 may include a syringe including a syringe housing 102 (or syringe barrel). The syringe housing 102 may have a cylindrical shape and an interior region or chamber within the syringe housing 102. The interior region may be described as having a longitudinal axis A1 extending from a first end 104 of the syringe housing 102 to a second end 106 of the syringe housing. In examples, the syringe can comprise a fitting or connector 108 disposed proximate the first end 104 of the syringe housing 102. In examples, the syringe may include a LUER-LOK® (Becton, Dickinson and Company, Corp., Franklin Lakes, N.J.) type connector 108 disposed proximate the first end 104 of the syringe housing 102. In examples, the connector 108 is configured to receive a needle, alternative cannula, tubing and/or adapters which can be utilized, for example, during administering of a medicant from within the interior region to an individual or in particular instances, for transferring into a distinct container (not shown). In examples, the first end 104 of the syringe housing 102 includes a male Luer-lock type connector to allow connection with a female Luer-lock fitting comprised by an administration needle. In examples, the first end 104 includes a female Luer-lock type connector.

In examples, a first end 104 of the syringe housing 102 may include a cap configured to seal the first end 104. The cap can prevent fluid from leaking from within the syringe housing 102 during storage, shipping, mixing, etc., and can prevent exposure of medicant components to an environment external to the syringe device. The cap can be configured to be reversibly attached by Luer-lock or other fitting mechanism, to allow removal and replacement by an appropriate administration or transfer attachment.

In examples, the syringe housing 102 may be a conventional type syringe barrel or may be manufactured for a particular application in accordance with the disclosure. The syringe housing 102 may be fabricated to comprise, for example, medical grade/approved glass or plastic material(s). Exemplary materials which can be utilized for the syringe housing formation include, but are not limited to, polyethylenes, polypropylenes, polycycloolefins, polyvinyl chloride (PVC), polyamides (including aliphatic and aromatic variants), polyesters, polycarbonates, copolymer materials including but not limited to those containing ethylene-diene-propylene monomer (EPDM), polyacrylates, polyurethanes, composites, blends or combinations of such materials, or alternative composite materials. The volume of the syringe (or alternate container) is not limited to a particular value and the syringe housing can be configured to contain a maximum volume of, for example, from 1 ml to greater than 10 ml. In examples, the syringe volume will be less than or equal to 10 ml. For purposes of the present description, the syringe volume refers to the volume of liquid which the syringe housing is configured to retain and not the overall volume within the interior region.

The mixing and/or administration system 100 can include a piston 110. The piston 110 may have a first end portion 112 and a second end portion 114 opposite the first end portion 112. The piston 110 includes a fluid passageway that extends longitudinally through the piston along a longitudinal axis A1 from a first end of the piston 110, through the piston 110, and out a second end such that fluid communication can be established between the vial 126 and the syringe housing 102. Activating a valve (e.g., twisting of a twist valve or pushing on a push-push valve 118) coupled to the piston 110 enables fluid to flow through the piston 110 passageway and allows fluid communication between the syringe housing 102 and the vial 126.

In examples, the piston 110 can comprise a valve opening extending through the piston 110. In examples, the valve opening may be located proximate the second end of the piston. The valve opening may orthogonally intersect the fluid passageway. Such valve opening can be configured to allow insertion of a valve (e.g., push-push valve 118). The positioning of the valve is not limited to any particular location and can be anywhere along the length of the piston 110. This positioning can advantageously allow ease of manipulation of the valve through a packaging surrounding the mixing and/or administration system 100.

In examples, the piston 110 may be manufactured as a single piece by, for example, injection molding techniques. In examples, the piston 110 may include a stopper 113 located at a first end portion 112 of the piston 110. In examples, the stopper 113 can be provided as an independent component or can be an integral part of the piston 110. In examples, the stopper 113 can comprise a material that differs from the piston 110 or can be formed of a material identical to the material utilized for forming the piston 110. The stopper 113 may be configured to have an opening or channel passing entirely through the stopper 113 and allowing fluid passage from within the passage through the stopper 113. The first end portion 112 of the piston 110 is configured such that the first end portion 112 is receivable within the syringe housing 102 such that fluid communication can be established between the syringe housing 102 and the piston passageway through the stopper 113 via an opening.

The piston 110 and the syringe housing 102 portions of the mixing and/or administration system 100 may comprise standard materials utilized for conventional syringe and piston/plunger formation. In examples, the piston 110 may comprise a relatively hard plastic. In embodiments where the stopper 113 is integral with the piston 110, the integrated piece may be formed of a common plastic material. Exemplary plastics which can be utilized for piston formation include but are not limited to polyethylenes, polypropylenes, polycycloolefins, polyvinyl chloride (PVC), polyamides (including aliphatic and aromatic variants), polyesters, polycarbonates, polyacrylates, polyurethanes, copolymers, blends, composites, and combinations thereof.

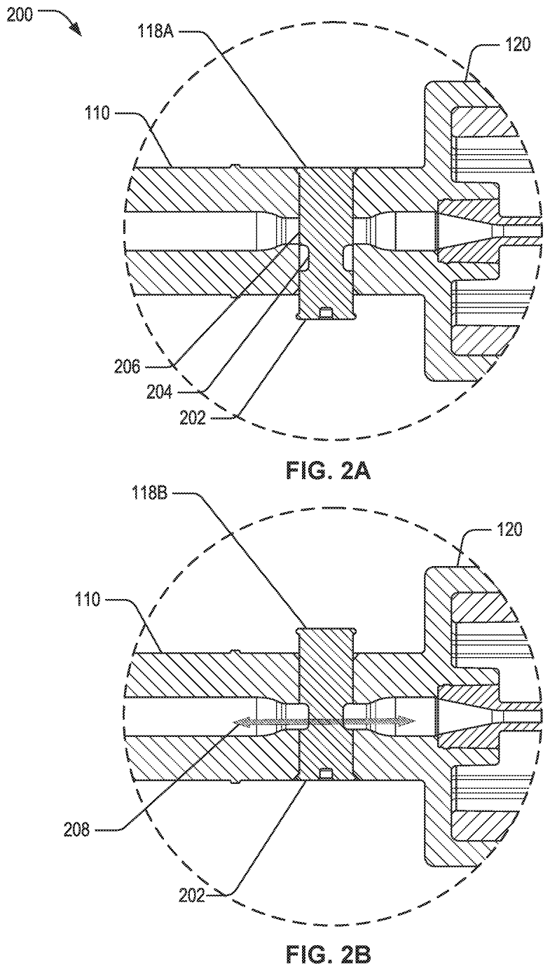

The mixing and/or administration system 100 may include a valve coupled to the piston 110. For example, the valve may be inserted into a valve opening located on the piston 110. In examples, the valve may be a push-push valve 118, a push-pull valve, or a twist valve. The push-push valve 118 is discussed in greater detail below in relation to FIGS. 2A and 2B.

The second end portion 114 of the piston 110 may include a fitting configured to accept or receive an extension housing 120. In examples, the extension housing 120 may have a uniform or non-uniform outer circumference. The extension housing 120 may have a receiving end 122 (or open end) configured to receive a vial 126. In some instances, the receiving end 122 of the extension housing 120 may have an internal diameter equal to or slightly smaller than the diameter of a vial 126 prior to insertion of the vial 126. In examples, insertion of a vial 126 can expand the receiving end 122 of the extension housing 120 due to the presence of slots on the extension housing 120. In examples, the extension housing 120 may be shaped to help retain the vial 126 within an internal area or receiving end 122 of the extension housing 120 until force is applied to extract the vial 126.

The piston 110 may be associated with a puncture device 124. The base of the puncture device 124 may be seated within a terminal portion of passageway of the piston 110 (e.g., the puncture device 124 may be seated proximate a second end of the piston 110). The piston 110 may have include a raised ridge 116 present proximate the second end portion 114 of the piston 110. In examples, the raised ridge 116 may prevent the syringe housing 102 from contacting the push-push valve 118 when the piston 110 is inserted into the syringe housing 102. In examples, the raised ridge 116 may extend around the entire perimeter of the piston 110 or partially around the perimeter of the piston 110. In examples, the piston 110 may include multiple raised ridges.

In examples, the puncture device 124 located at the second end portion 114 of the piston 110 may have a head portion comprising a tip disposed at a first end. The puncture device 124 may have a stem/body portion extending from the head portion to a base surface disposed at a second end of the puncture device 124 opposite the first end. A channel or other fluid passage may extend through the base surface and through the entire body portion of the puncture device 124. In examples, the channel or fluid passage may extend less than an entirety of an internal length of the head portion such that the channel does not pass through the tip disposed at the first end of the puncture device 124. The head portion may include a hole to allow fluid to pass through. The head portion of the puncture device 124 may include a cutting edge in order to help pierce the cap of the vial 126. The puncture device 124 may be formed from metals, such as stainless steel or titanium, and/or various plastics such as polyamides, polyacrylates, polycarbonates, epoxies, polyurethanes, polysulfones, polytherimides, polypropylenes, copolymers, etc., in either thermoplastic or thermoset varieties.

The mixing and/or administration system 100 may include a vial 126. The term “vial” is not limited to a particular container structure and can be used to refer to various containers including containers utilized for parenteral as well as non-parenteral materials. In examples, the vial 126 may be a bottle such as illustrated in FIG. 1A having a cap. The vial 126 may be a glass bottle or alternatively can be a plastic container or other material utilized conventionally or yet to be developed for retaining and/or accessing a medicant or component thereof. In examples, the vial 126 may be a standard medicant type vial. In examples, the extension housing 120 may be configured such that the vial 126 is slidably insertable into a receiving end 122 or an internal area of the extension housing 120. In examples, the vial may be coupled (e.g., screwed onto, clamped onto, etc.) to a portion of the extension housing 120. In examples, a first component can be provided within the vial 126 and a second component can be provided within the syringe housing 102. The syringe housing 102 may be capped to retain the second component, with such cap being reversibly attached to allow removal as appropriate.

As mentioned above, the mixing and/or administration system can be described as being a closed-system in that separate components can be combined and mixed without exposing the components to an environment external to the system. The mixing and/or administration system can be assembled in a sterile and clean environment (e.g., a pharmaceutical company, clean room, or other highly controlled room environments). During assembly of the mixing and/or administration system, the correct components are paired together and provided in the proper amounts within the mixing and/or administration system, all within a sterile environment. This ensures that individuals using the assembled mixing and/or administration system are not required to match pairs of components and separately draw a correct amount of each under a laminar flow hood (which may expose the medicants to contaminants in the air) or in an unsterile environment (e.g., in a clinic, a patient's home, in an emergency situation, etc.).

A general methodology in accordance with the mixing and/or administration system 100 can comprise mixing a first component provided within the vial 126 with a second component provided within the syringe housing 102. In an initial state prior to the combining of the two components, each component is isolated from the other. The piston 110 may be provided in an initial position relative to the syringe housing 102 with the push-push valve 118 being initially disposed in an “off” position and blocking fluid passageway through the piston 110.

In examples, the mixing and/or administration system 100 may include a syringe housing 102 that is capped or otherwise sealed to prohibit passage of material into or out of the syringe housing 102 through the first end of the syringe. In examples, the piston 110 may initially be disposed in an inserted position through the second end of syringe housing and positioned to allow containment of the second component within the syringe housing. While the push-push valve 118 is in the off position, the vial 126 may be partially inserted within the extension housing 120. It is to be noted that such insertion can, in some examples, be performed by an end user of the mixing and/or administration system 100 or can be performed prior to packaging of the mixing and/or administration system 100. Regardless, the vial cap/septum is initially provided to be intact and preferably to be spaced from the puncture device 124 such that the tip of the puncture device 124 is not in physical contact with any portion of the vial 126 as initially provided.

Once combination and mixing of the separate components is desired, the vial 126 can be repositioned by, for example, sliding the vial 126 farther within the extension housing 120 to allow the puncture device 124 to puncture the cap of the vial 126 and be partially inserted through the septum or alternate barrier portion of the vial 126. Once puncturing has occurred, the push-push valve 118 can be pushed or otherwise repositioned into an open position allowing fluid passage through the piston 110. Such repositioning establishes fluid communication between the interior of the vial 126 and the interior of syringe housing 102 without exposing either of the two components to an environment external to the mixing and/or administration system 100. In examples, the first and/or second component may be corrosive, toxic, and/or an irritant. In examples, the first and/or second component may be a chemo drug. In examples, one or both of the first component and second component may be in liquid form. In examples, the component within the syringe housing 102 may be in liquid form and the component within vial 126 may be in a dry, powdered, or lyophilized form, but may alternatively be in the form of a liquid, solution, suspension or other mixture. In examples, where the vial 126 contains a non-fluid component, a liquid component contained within syringe housing 102 may be introduced into the vial 126 and can be combined with the component within the vial 126 by, for example, sliding the piston 110 from an initial position to a second position such that the stopper 113 of the piston 110 is repositioned to be closer to the first end 104 of syringe housing 102. Such sliding motion allows fluid to flow from within the syringe housing 102 through the piston 110 and into the vial 126 via an opening or channel created by the activated push-push valve 118 (or in the “open” position).

In examples, mixing of the combined components can be performed by a forward and reverse sliding motion of piston 110 relative to the syringe housing 102 in a “pumping” type motion. The pumping motion is conducted with the push-push valve 118 in the open position allowing fluid communication between the syringe housing 102 and the vial 126, typically with the vial 126 being in an inverted position. Alternatively, mixing can be conducted by shaking or agitation of the vial 126 and/or the entire mixing and/or administration system 100, or by a combination of pumping action and shaking, agitating, etc. Once the components have been mixed, the mixing and/or administration system 100 may be prepared for transfer of the mixed agent or, where the mixture is an administration-ready agent the syringe can be prepared for administrating the agent to an individual. Alternatively, if additional components are to be combined with the mixture, such can be introduced by, for example, flowing into the syringe housing 102 via the first end 104, and mixing as described above.

In order to prepare for transfer and/or administration of the agent, the mixture can be drawn into a syringe housing 102 by, for example, sliding piston 110 in a rearward motion, typically with the vial 126 in an inverted position. In other words, the piston 110 is partially extracted and the stopper 113 is moved toward the second end 106 of the syringe housing 102. During the drawing of all or an appropriate measured amount of the mixture into the syringe housing 102, the push-push valve may be in the closed position and blocking fluid passageway from the syringe housing 102 through the piston 110. The syringe housing 102 can then be uncapped by, for example, removal of a cap such as a Luer-lock cap fitting. It is to be understood that the mixing/preparation of the medicant may include a needle fitted to a Luer-lock fitting during the preparation stage. However, the needle is preferably capped and sealed during such operation or otherwise prevented from allowing passage or exposure of material from the syringe housing 102 to an environment external to the mixing and/or administration system 100.

Where a cap is removed in preparation for transfer or administration of the agent or component, an appropriate transfer device such as a needle, cannula, transfer tube and/or other appropriate fitting can be attached to the Luer-lock connection and transfer/administration can occur by sliding forward of the piston 110 within the syringe housing 102, thereby expelling the contents of the syringe housing 102 through the needle or alternate transfer structure at the Luer-lock end of the syringe housing 102 (i.e., at a first end 104 of the syringe housing 102). Such transfer is performed with the push-push valve 118 remaining in an un-activated state (e.g., a closed position) throughout.

FIG. 2A is a cross-sectional view 200A of a push-push valve in a closed position 118A in accordance with an example of the present disclosure. In examples, the push-push valve 118 may have a head portion 202, a neck portion 204, and a body portion 206. The head portion 202 may be configured to have an extension or protrusion tab that extends from one or more sides of the head portion 202. Any number of extension tabs can be utilized. In examples, the extension tab may assist in proper positioning and alignment of a fluid passageway 208 which passes through a neck portion 204 of the push-push valve 118. The head portion 202 of the push-push valve 118 may have a circular, square, hexagonal shape, or any other shape. In examples, the head portion 202 may have an arrow-shape to allow visual and/or tactile indication of the push-push valve's position. The head portion 202 may also be configured to have alternative or additional visual and/or tactile indicators.

The body portion 206 of the push-push valve 118 may be positioned within the piston 110 so that no fluid can pass through the push-push valve 118 while the push-push valve is in a closed position 118A. In examples, one or more seals or o-rings may be provided to create a fluid seal within the opening in the neck portion 204. In examples, the opening in the neck portion 204 may have a uniform-diameter cylindrical shape, conical shape, rectangular shape, or other shape. In examples, the body portion 206 may be appropriately shaped to be received within a valve opening in the piston 110.

The push-push valve 118 may not be limited to a particular material and can comprise plastic and/or elastomeric materials. In examples, the body portion 206 comprises an elastomeric material to allow a better fit and/or seal within the valve opening of the neck portion 204. Elastomeric materials may include, but are not limited to, polyurethanes, polypropylene-EPDM, other polypropylenes, polysiloxane and/or silicone materials, butyl materials, isoprenes, neoprenes, polyethylenes, and various copolymers, composites, blends or other combinations of such materials. Additional appropriate materials may include natural rubbers, nitrile rubbers and combinations thereof. In examples, the push-push valve 118 may be constructed as a single piece and may be formed of a particular material. In examples, the head portion 202 may be formed independently of the neck portion 204 and/or the body portion 206 and may comprise of a material that differs from the neck portion 204 and/or body portion 206. For example, the head portion 202 may be formed of a hard plastic such as any of those listed above and the body portion 206 may comprise either a distinct hard plastic material or any of the elastomeric materials listed above.

FIG. 2B is a cross-sectional view 200 of a push-push valve 118 in an open position 118B in accordance with an example of the present disclosure. When a user pushes on the head portion 202 of the push-push valve, the neck portion 204 aligns with the piston and the puncture system and a fluid passageway 208 is created. That is, activating the push-push valve causes a channel to open between the syringe housing and the extension housing including the vial. The fluid passageway 208 allows for medicinal agents or components to flow to and from the vial. In examples, the push-push valve 118 on the piston 110 may be a “one-time” use valve such that once the push-push valve 118 is activated or pushed, a user is not able to push the push-push valve 118 in the opposite direction and close the opened passageway. In some examples, the push-push valve 118 is configured to be bi-directional such that the passageway between the vial and the syringe housing may be opened and closed at will.

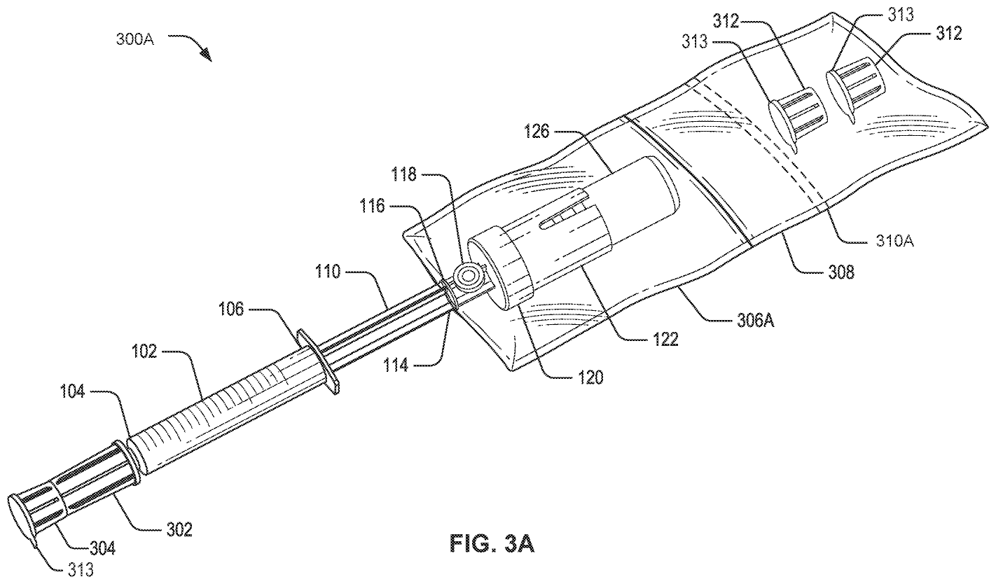

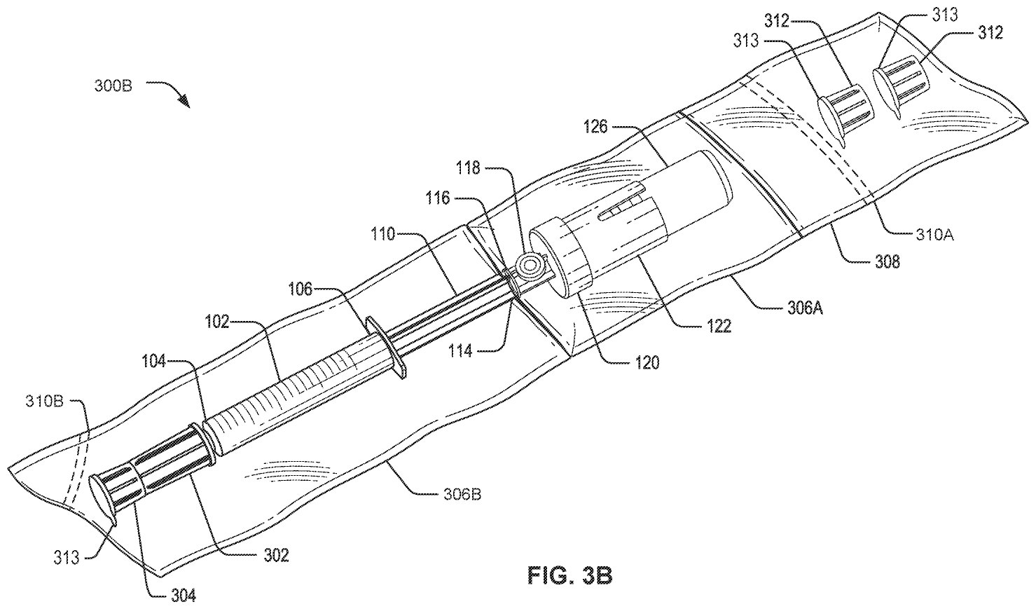

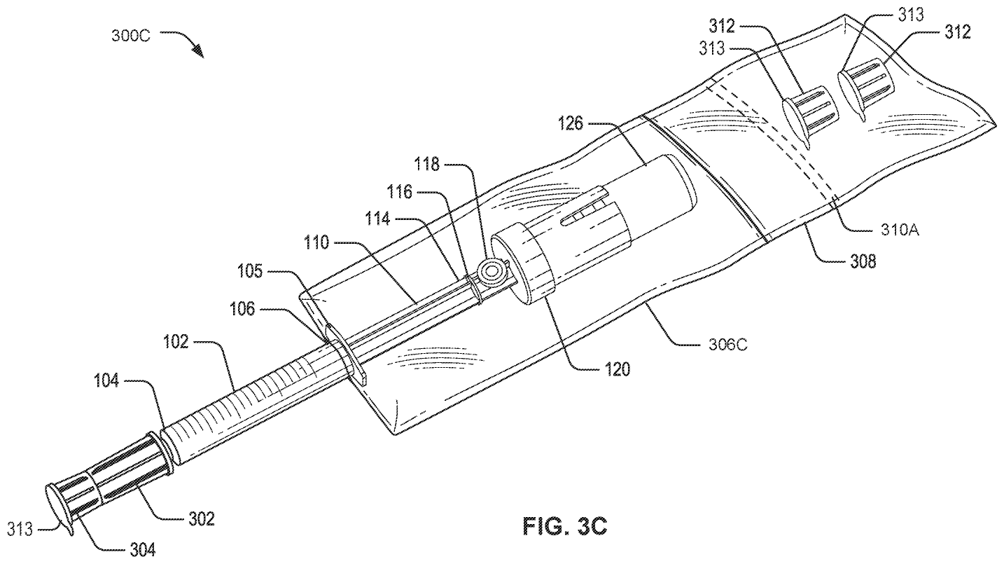

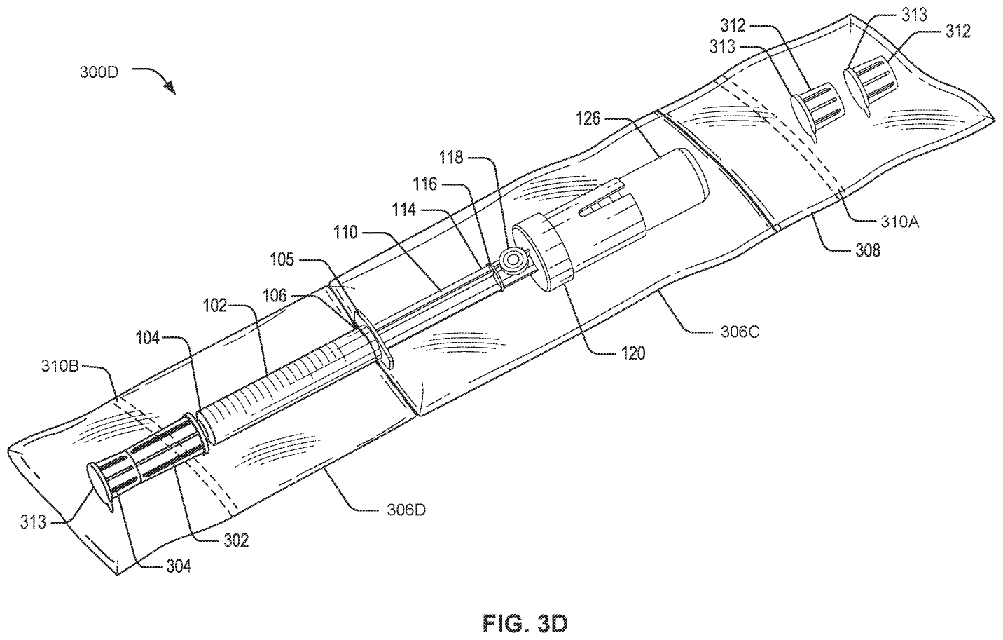

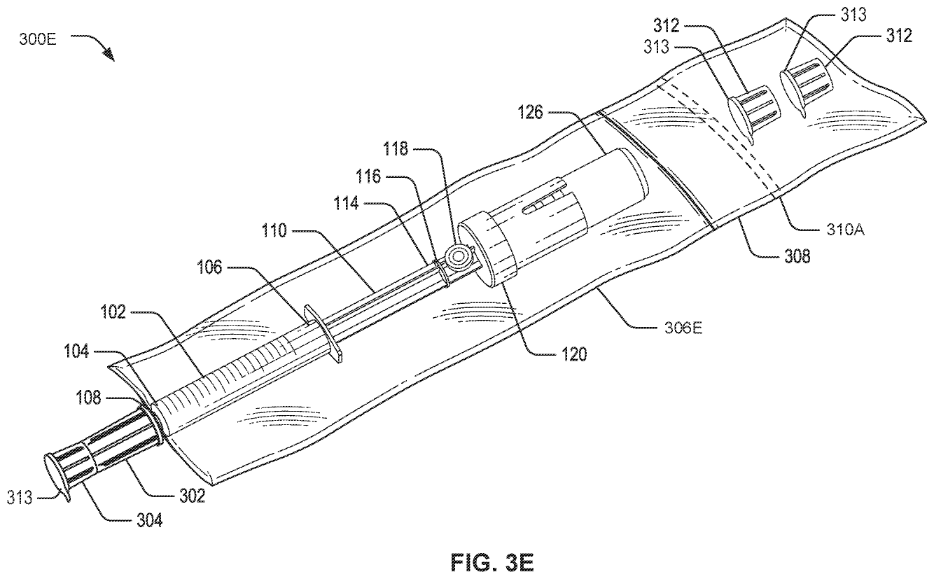

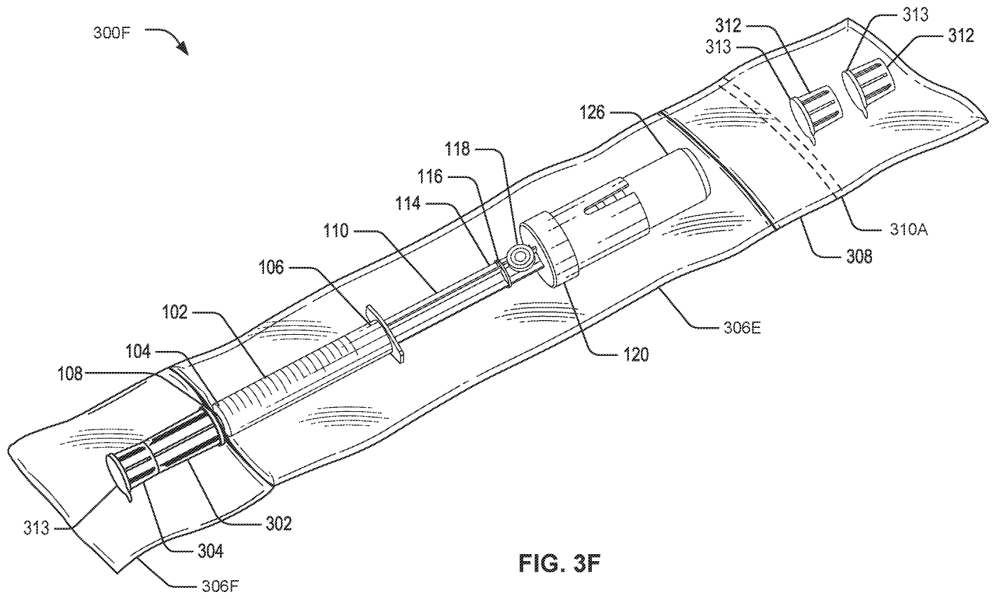

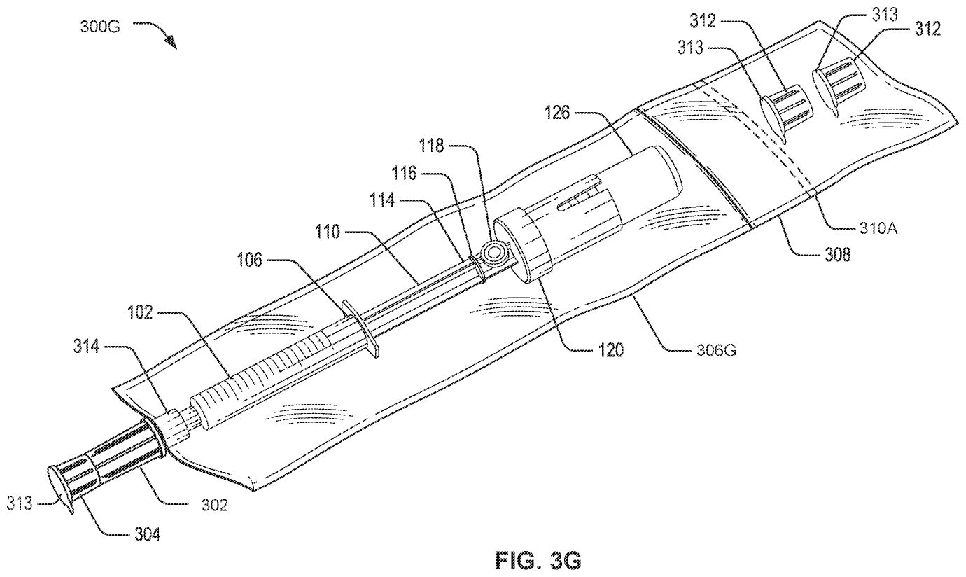

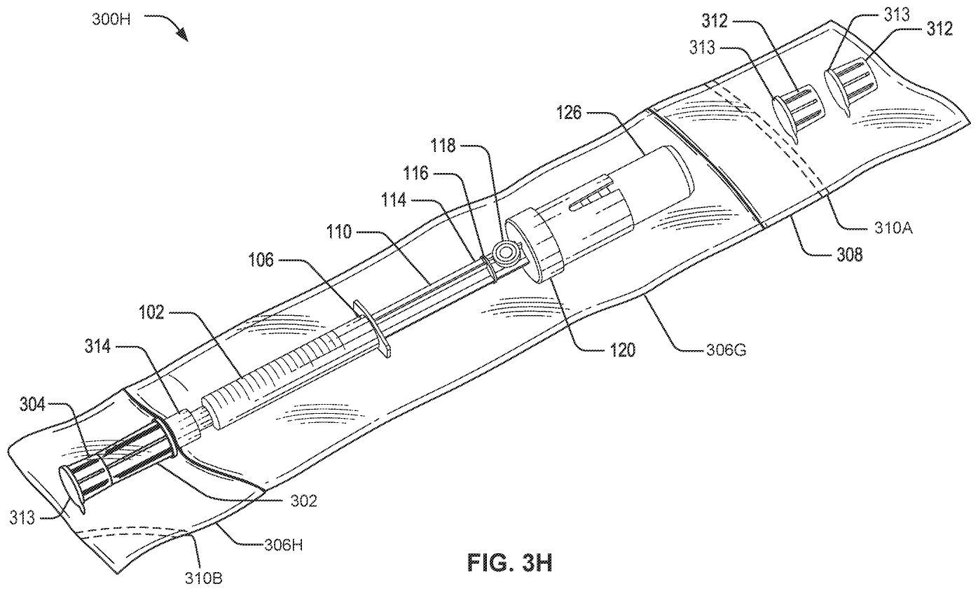

FIG. 3A is a perspective view of a mixing and/or administration system 300A including packaging 306A sealed to a portion of a piston 110 in accordance with an example of the present disclosure. In examples, the packaging may be sealed around the entire mixing and/or administration system or a portion of the mixing and/or administration system so that the one or more portions of the mixing and/or administration system is maintained in a sterile environment. In examples, the packaging 306A may be formed from a transparent or translucent material. In examples, a portion of the packaging may be transparent. For example, a first side of the packaging 306A may be transparent and a second side opposite the first side may be opaque. In examples, the packaging 306A may be formed from a flexible material so that a user may easily manipulate the push-push valve and mix components within the mixing and/or administration system without opening the packaging.

In examples, the packaging 306A may be formed from a material capable of creating a moisture and air barrier. In examples, the packaging 306A may be formed from a corrosion resistant material. In examples, the packaging 306A may be formed from a material resistant to hydrogen peroxide, alcohols (e.g., small molecule alcohols such as methanol, ethanol, isopropyl alcohol, etc.), ethylenediaminetetraacetic acid (EDTA), and the like. In examples, the packaging 306A may be formed from a medical grade, flexible thermoplastic polyurethane material. In examples, the packaging 306A may be formed from one or more of a modified polyethylene terephthalate glycol (PETG) material, a high-impact polystyrene (HIPS) material, glycol modified polyethylene terephthalate foam (PETG foam), polypropylene (PP), polycarbonate, amorphous copolyester, high-density polyethylene, nylon/Tyvek, polyvinyl chloride (PVC) cellulose acetate, medical grade paper, and the like. In some examples, the packaging may be formed by multiple layers of material, which collectively provide the desired material properties (e.g., structural strength, transparency, and air and moisture barrier). In some examples, an interior and/or exterior surface of the packaging 306A may be coated with an additional moisture and air barrier layer or material which can be applied by, for example, a roll bonding process or a deposition process. In some examples, the packaging 306A may be coated (in whole or in part) in an anticorrosion material.

In the particular example, the packaging 306A surrounds a portion of the mixing and/or administration system 300A including the vial 126, the extension housing 120 and the push-push valve 118. The packaging 306A may be sealed or coupled to a portion of the piston 110 using a heat staking process, sonic welding, a medical grade adhesive, and/or other bonding techniques. In examples, the packaging 306 may be sealed to the raised ridge 116 located proximate the second end portion 114 of the piston 110. In examples, the packaging may be sealed to a portion of the push-push valve 118, the extension housing 120, and/or the vial 126.

In examples, the first end 104 of the syringe housing 102 may include a protective cap 302 coupled to the tip of the syringe. In some examples, the disinfecting cap 304 may include a removable lid 313 (e.g., a lid with a pull tab, a cap, etc.) configured to protect the contents of the disinfecting cap 304. In some examples, the removable lid 313 may be coupled to the disinfecting cap 304 via any suitable manner (e.g., via adhesive, chemical bonding, ultrasonic welding, using heat sealing techniques, or any other method known in the art). In examples, a disinfecting cap 304 may be separately sealed and coupled to an end of the protective cap 302. For example, a base portion opposite the removable lid 313 of the disinfecting cap 304 may be coupled to a base portion of the protective cap 302. In examples, the mixing and/or administration system 300A may include additional disinfecting caps. For example, a separate, sealed pouch 308 may be coupled to the packaging 306A via a hermetic seal. The separately sealed pouch 308 may include one or more cleaning devices (e.g., the sealed pouch 308 may include a pair of disinfecting caps, one or more sponge applicators, alcohol wipes, swabs, etc.). In some examples, the separately sealed pouch 308 may include individual disinfecting caps separately sealed in their own sterile packaging that includes an antimicrobial or cleaning composition. In some examples, individual disinfecting caps may include a removable lid 313 with a pull tab, cap, etc. In some examples, the antimicrobial or cleaning composition may include isopropyl alcohol, hydrogen peroxide, iodine, a chelator (e.g., EDTA), water, citrate (including the salt forms of citrate such as monosodium citrate, disodium citrate, and trisodium citrate), a biocompatible cleanser, a combination of any of these, or any other natural or synthetic antimicrobial or cleaning composition. In some examples, the packaging may include an antimicrobial composition such as any of those described in U.S. Pat. No. 9,253,987 to Tennican et al., filed Jul. 2, 2013, which is incorporated herein by reference.

In examples, the separately sealed pouch 308 may include a tear strip 310A configured to provide access to the cleaning device without exposing the vial to an external environment outside the packaging. In examples, the tear strip 310A may extend across an entire width or length of the separately sealed pouch (e.g., from a first side to a second side of the sealed pouch) or partially across less than all of a length or width of the separately sealed pouch 308.

A disinfecting cap 312 may be configured to fit and disinfect both male and female Luer connectors as well as other connectors such as IV connectors, catheters (e.g., hemodialysis catheters, peritoneal dialysis catheters, subcutaneous catheters, etc.) and other medical devices. In examples, a disinfecting cap 312 may be configured to fit and disinfect medical tubing, and/or medical devices used in administering intradermal injections, subcutaneous injections, vaccines, and the like. Disinfecting cap(s) included with the mixing and/or administration system may be of various shapes, sizes, types, may be used for various purposes, and/or may be produced by different manufacturers, etc. For example, individual disinfecting cap(s) 312 may be configured to fit a male Luer connector, a female Luer connector, or may be configured to fit both a male and female Luer connector or port. In examples, a first disinfecting cap may be configured to fit and disinfect a male Luer connector and a second disinfecting cap may be configured to fit and disinfect a female Luer connector. In examples, the sealed pouch may include a disinfecting cap and another cleaning device, such as a sponge, swab, alcohol wipe, etc. In some examples, the mixing and/or administration system 300A may include multiple, separately sealed pouches that house any number of cleaning devices (e.g., sponge applicators, gauze, alcohol prep pads, and/or cotton swabs). In examples, the separately sealed pouch may include a separate tear strip that provides a user with easy access to the separately sealed pouch without exposing the other components of the mixing and/or administration system (e.g., the vial and syringe housing) to the environment.

FIG. 3B is a perspective view of the mixing and/or administration system 300B in FIG. 3A including a second packaging 306B sealed to the portion of the piston. The second packaging 306B may comprise a separately sealed pouch that protects the piston 110, syringe housing 102, and end of the syringe from contaminants that may be present in the environment external to the mixing and/or administration system. The second packaging 306B may include a second tear strip 310B configured to provide access to the syringe tip. In examples, the tear strip 310B may extend across an entire length of a corner of the second packaging 306B (e.g., from a first side to a second side of the second packing) or partially across a length of the corner.

FIG. 3C is a perspective view of a mixing and/or administration system 300C including packaging 306C sealed to a syringe housing 102. In the particular example, the packaging 306C surrounds the vial 126, the extension housing 120, the push-push valve 118, the piston 110, and is sealed or coupled to a portion of the syringe housing 102. In examples, the packaging 306C may be sealed to a second end 106 of the syringe housing 102, proximate the syringe flanges 105 located on the syringe housing 102. In examples, the packaging 306C may be at least partially sealed to the syringe flanges 105 (e.g., sealed to a side of the syringe flanges 105 facing the syringe housing 102). In examples, sealing a portion of the packaging 306C to one or more portions of the mixing and/or administration system 300C enables a user to manipulate contents of the mixing and/or administration system more easily (e.g., the push-push valve 118 may be easier to activate).

FIG. 3D is a perspective view of the mixing and/or administration system 300D including a second packaging sealed 306D to a syringe housing 102 and surrounding the syringe housing 102, protective cap 302, and disinfecting cap 304. In examples, the second packaging 306D may be sealed (in whole or in part) to the packaging 306C. The second packaging 306D may comprise of a medical grade/approved plastic material, as discussed throughout the disclosure. The second packaging 306D may include a second tear strip 310B configured to provide easy access to the syringe tip. In examples, the tear strip 310B may extend across an entire length of the second packaging 306B (e.g., from a first side to a second side of the second packing) or partially across a length of the second packaging.

FIG. 3E is a perspective view of a mixing and/or administration system 300E including packaging 306E sealed or coupled to a connector 108. In examples, the packaging 306E may be sealed to a first end 104 of the syringe housing 102. In examples, the packaging 306E may be sealed to a portion of the protective cap 302 coupled to the connector 108.

FIG. 3F is a perspective view of the mixing and/or administration system 300F including a second packaging 306F sealed to the connector 108 and surrounding the protective cap 302 and disinfecting cap 304. In examples, the second packaging 306F may be sealed (in whole or in part) to the packaging 306E. The second packaging 306F may comprise of a medical grade/approved plastic material, as discussed throughout the disclosure.

FIG. 3G is a perspective view of a mixing and/or administration system 300G including packaging 306G sealed to a Luer connector 314. In examples, the Luer connector 314 may be used to prevent fluid from leaking when fluid is transferred from the mixing and/or administration system 300G. In examples, the Luer connector 314 may be a double ended Luer connector. In examples, the Luer connector 314 may be a male or female Luer connector and may be configured to connect to IV tubing or other external Luer IV connectors. In examples, a first end of the Luer connector 314 may be coupled to a connector associated with the syringe housing 102 and a second end opposite the first end of the Luer connector 314 may be covered by a protective cap 302. In the particular example shown in FIG. 3G, a portion of the packaging 306G may be coupled to the Luer connector 314. In examples, a portion of the packaging 306G may be coupled to a protective cap 302 that is coupled to the Luer connector 314.

FIG. 3H is a perspective view of a mixing and/or administration system 300H including a second packaging 306H sealed to a Luer connector 314 and surrounding the protective cap 302 and disinfecting cap 304. In examples, the second packaging 306H may be sealed (in whole or in part) to the packaging 306G. The second packaging 306H may include a second tear strip 310B configured to provide easy access to the syringe tip. In examples, the tear strip 310B may extend across an entire length of the second packaging 306H (e.g., from a first side to a second side of the second packing) or partially across a length of a corner.

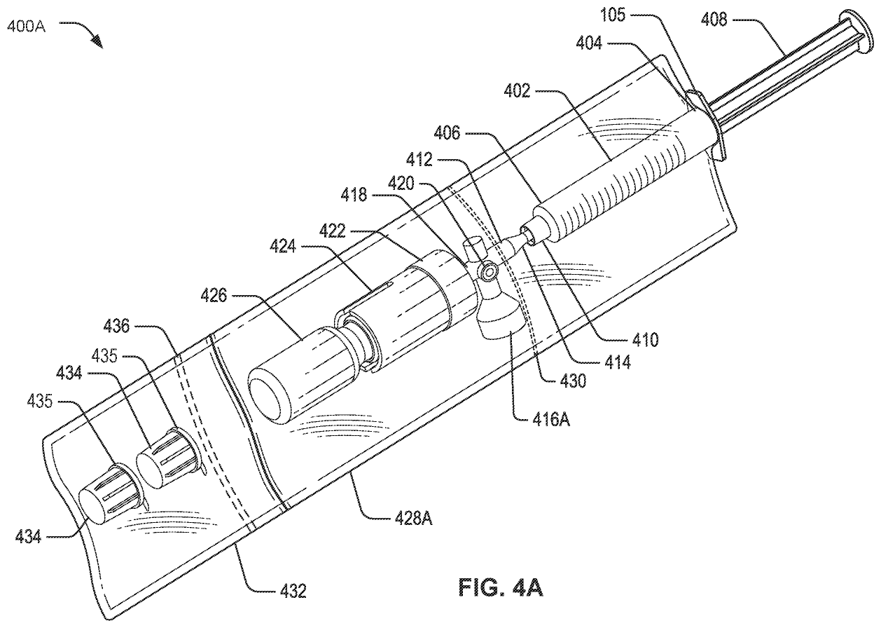

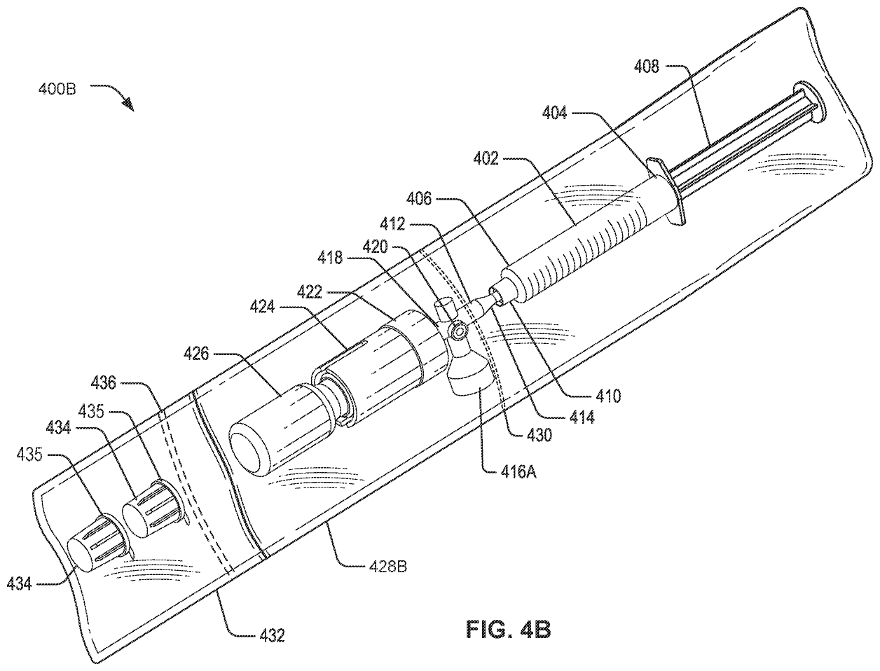

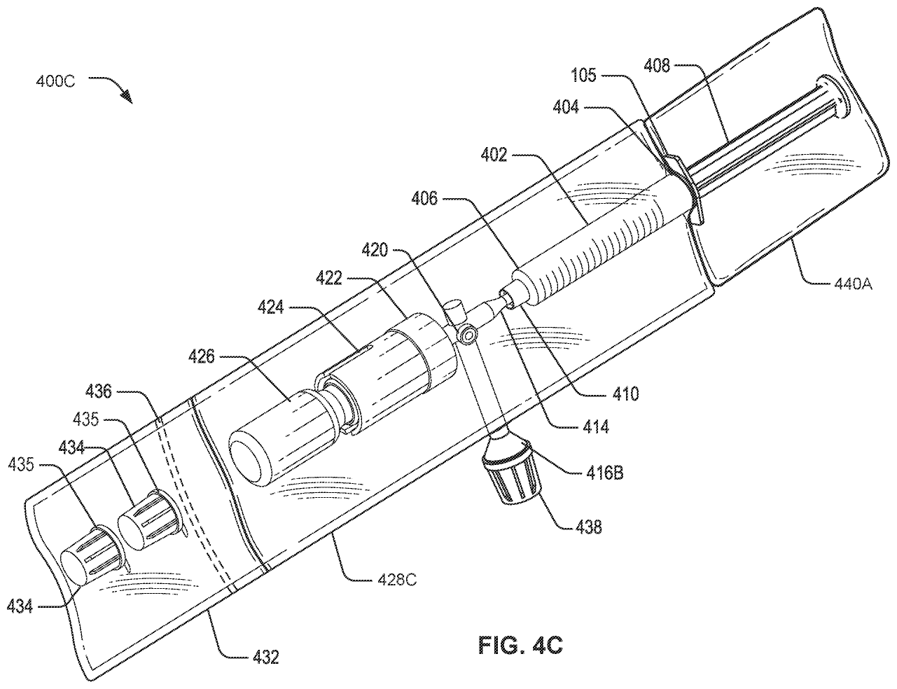

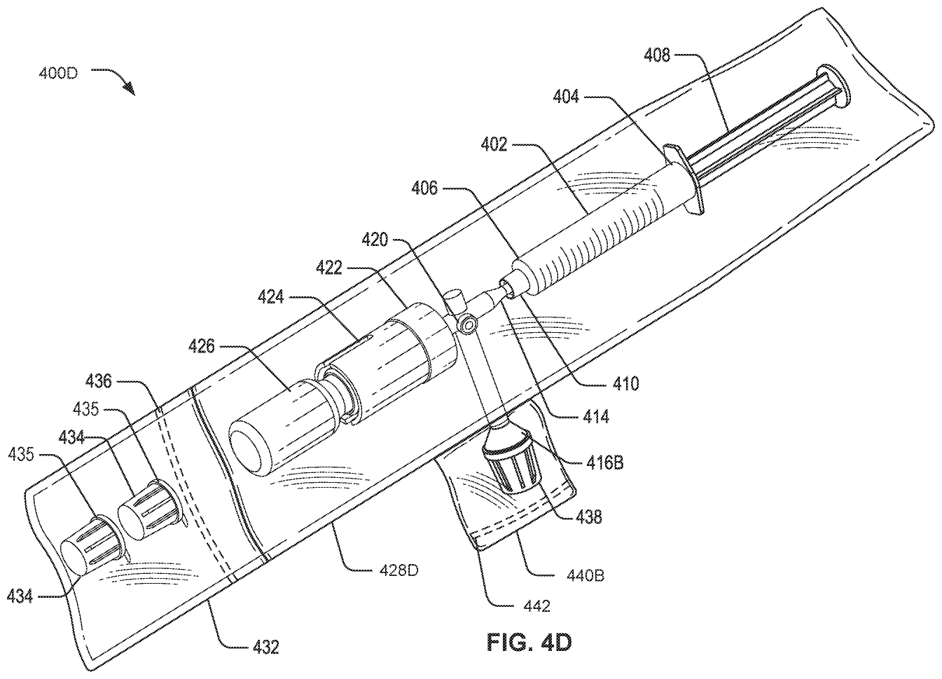

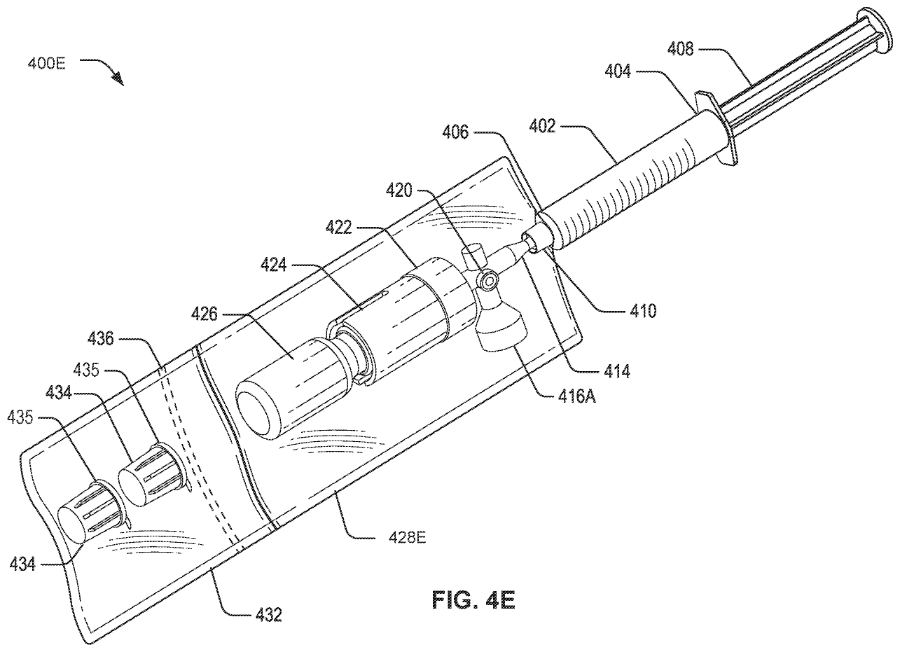

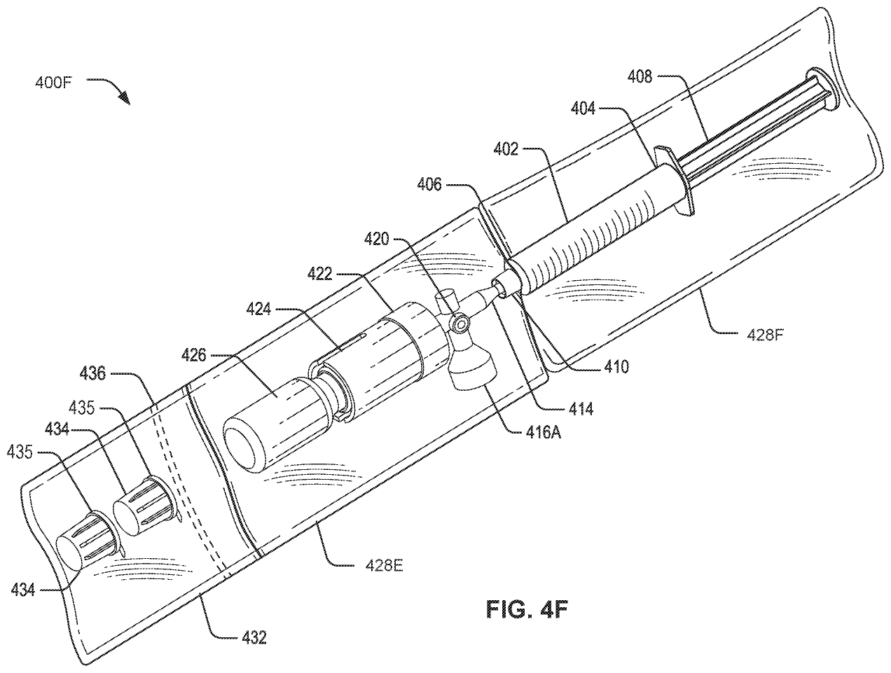

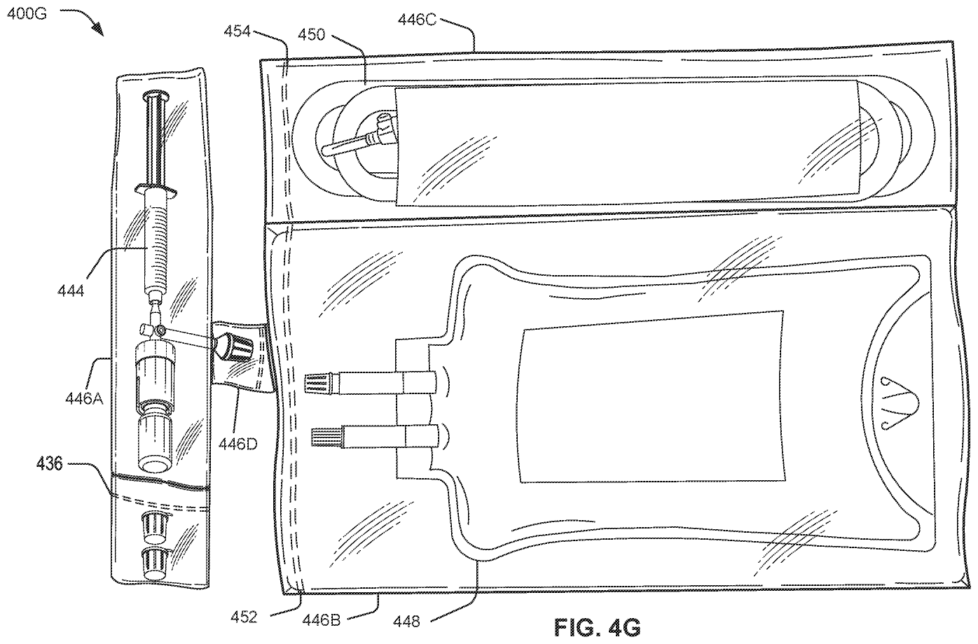

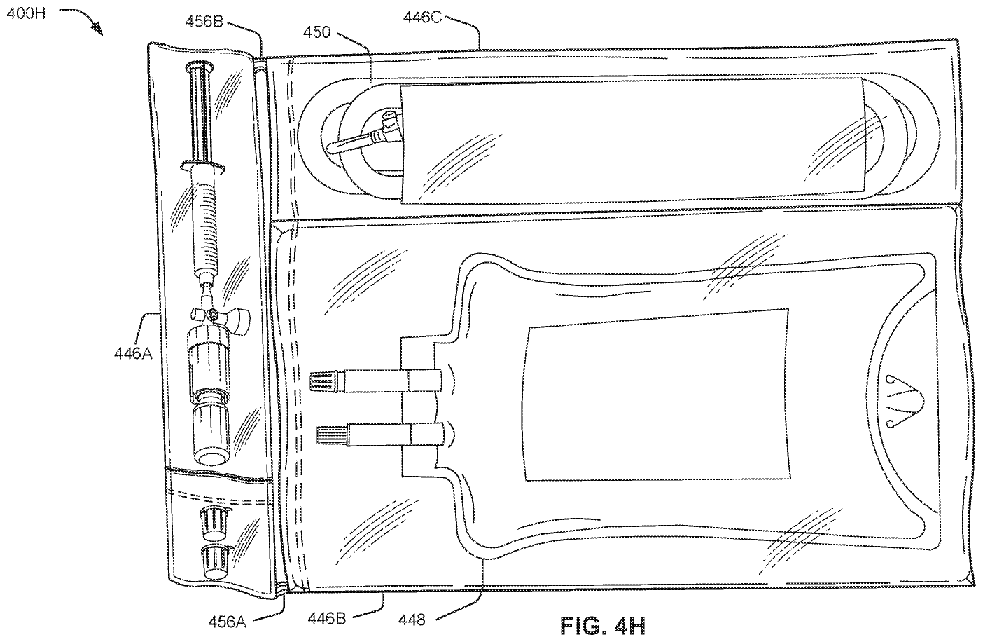

FIG. 4A is a perspective view of an alternative mixing and/or administration system 400A including packaging sealed to a syringe housing. A mixing and/or administration system 400A can comprise a syringe housing 402 having a first end 404 and a second end 406. A piston 408 may be slidably coupled to the first end 404 of the syringe housing 402 and a Luer-lock fitting 410 may be coupled to the second end 406 of the syringe housing 402. In examples, the Luer-lock fitting 410 may be coupled to an adapter 412. In examples, the adapter 412 may be reversibly attachable to the syringe housing 402 by a female Luer-lock fitting or alternative fitting on a first end of the adapter 412. In examples, the Luer-lock fitting 410 may be configured to be removed from the adapter 412 and coupled to a transfer device such as a needle, cannula, transfer tube or other transfer structure that can be attached to the Luer-lock fitting 410.

In examples, the adapter 412 may be formed of any appropriate material such as any of the plastic materials discussed above with reference to the syringe housing and piston. In examples, the adapter 412 may be a “three-way” adapter having a first segment which extends from a three-way push-push valve 420 to a first port 414 having a fitting (for example a Luer-lock type fitting) for connection with the Luer-lock fitting 410 associated with the syringe housing 402. The adapter 412 additionally includes a second segment which extends from the position of the push-push valve 420 to a second port 416A. In examples, the push-push valve 420 may be positioned perpendicular or oblique to the second port 416A. The adapter 412 additionally has a third segment extending from a position of the push-push valve 420 to a third port 418 which can comprise a fitting configured for insertion or other association with an extension housing 422. The third port 418 can additionally be configured to include a puncture device (not shown) such as the puncture device described above in relation to FIGS. 1A and 1B. An adapter 412 including three ports allows introduction and/or removal of material to or from the mixing and/or administration system 400A without dissociation of the syringe housing 402 and/or the vial 426. For example, after mixing or preparing a medicant utilizing methodology analogous to that described above, the prepared medicant can be transferred or otherwise removed from the mixing and/or administration system 400A through the second port 416A. Such transfer or removal can be accomplished by, for example, attaching an appropriate vial, transfer tubing or other transfer apparatus to the second port 416A. In examples, the second port 416A may include a Luer-lock type fitting or other alternative fitting that can be utilized for attachment to a desired container or transfer apparatus. Transfer of material from the mixing and/or administration system 400A via the second port 416A may be useful for applications such as IV administration. A liquid material may be introduced into the mixing and/or administration system 400A using the fluid passage through the second port 416A and adapter 412. The introduced fluid can be directed into the vial 426 and/or into the syringe housing 402.

An extension housing 422 may be coupled to the adapter 412. The extension housing 422 may have any of the configurations described above for receiving, stabilizing and/or retaining the vial 426 which is shown in a partially inserted position within the open end 424 of the extension housing 422. In examples, the mixing and/or administration system 400A includes a vial 426 that is only partially inserted within the extension housing 422 so as to avoid the piercing structure.

In examples, the mixing and/or administration system 400A may be include a packaging 428A surrounding the mixing and/or administration system 400A. In the particular example, the packaging 428A surrounds a portion of the mixing and/or administration system 400A including the vial 426, the extension housing 422, the push-push valve 420, the adapter 412, the syringe housing 402, and the packaging is sealed to a first end 404 of the syringe housing 402. In examples, the packaging 428A may be sealed to the syringe flanges located at the first end 404 of the syringe housing 402. The packaging 428A surrounding the mixing and/or administration system 400A can be formed by, for example, a molding process. In examples, the packaging 428A may comprise of a plastic material having suitable strength for providing positioning and stability of the mixing and/or administration system 400A. In examples, the packaging 428A may comprise of a medical grade/approved plastic material, as discussed above. In examples, the packaging can allow for mixing and preparation of a medicant in a sealed environment. In examples, the prepared medicant can be transferred from the syringe housing 402 by attaching an appropriate transfer apparatus to the second port 416A. In examples, such attachment and transfer can be conducted while all or a portion of the mixing and/or administration system remains within the packing enclosure.

In examples, the packaging 428A may include a tear strip 430 positioned proximate the adapter 412. In examples, the tear strip 430 may be positioned proximate the second port 416A to allow for ease of access to the second port 416A. In examples, the tear strip 430 may extend across an entire length of the packaging 428A (e.g., from a first end to a second end of the packaging). In examples, the tear strip 430 may partially extend across a length of the packaging 428A. In examples, the tear strip 430 may be a U-shape tear strip and positioned proximate the second port 416A to provide ease of access to the second port 416A.

In examples, the packaging 428A may include a separate, tear open pouch 432 including at least one disinfecting cap 434. In examples, the disinfecting cap may be separately sealed with a removable lid 435 (e.g., a lid with a pull tab, a cap, etc.) configured to protect the contents of the disinfecting cap 434. In examples, the pouch 432 may be coupled to the packaging 428A via a tear strip 436. In examples, the pouch 432 may be attached to any side or portion of the packaging 428A.

In examples, the push-push valve 420 associated with the adapter 412 may be pushed or otherwise activated into an open position in order to establish fluid communication between a first fluid passageway associated with the syringe housing 402, a second fluid passageway associated with the second port 416A, and a third fluid passageway associated with the third port 418 proximate the extension housing 422. Activating the push-push valve 420 allows fluid to pass through the adapter 412 and establish fluid communication between the vial 426 and the syringe housing 402.

Methodology for mixing independent components or medicants to prepare a mixture or administration-ready agent utilizing the mixing and/or administration system 400A may comprise providing a first component in a vial 426 and providing a second component in to be combined and mixed with the first component within syringe housing 402. The syringe housing 402 and the vial 426 are each attached to the adapter 412. Attachment and puncturing of the vial 426 can be performed as described above with respect to earlier examples provided in relation to FIGS. 1A and 1B. In examples, the push-push valve 420 is in a closed position during attachments of the vial 426 and the syringe housing 402. In examples, the push-push valve 420 on the adapter 412 may be a “one-time” use valve such that once the push-push valve 420 is activated or pushed, a user is not able to push the push-push valve 420 in the opposite direction and close the opened passageway. In some examples, the push-push valve 420 is configured to be bi-directional such that the passageway between the vial 426 and the syringe housing 402 may be opened and closed at will.

Upon assembly, the push-push valve 420 may be pushed or otherwise manipulated into an open position in order to establish fluid communication between the vial 426 and the syringe housing 402 by allowing fluid passage through the adapter 412. Accordingly, fluid communication is established between the vial 426 and the syringe housing 402.

FIG. 4B is a perspective view of the alternative mixing and/or administration system 400B including a packaging 428B surrounding the mixing and/or administration system 400B. In examples, the packaging may be coupled (e.g., using heat sealing techniques, ultrasonic welding, adhesive bonding techniques, sonic welding, etc.) to portions of the syringe device (e.g., piston 408, syringe flanges, syringe housing 402, or any other portion of the mixing and/or administration system described herein).

FIG. 4C is a perspective view of an alternative mixing and/or administration system 400C having a second port 416B coupled to the packaging 428C in accordance with an example of the present disclosure. In the particular example, the packaging 428C surrounds a portion of the mixing and/or administration system including the vial 426, the extension housing 422, the push-push valve 420, a portion of the adapter 412, the syringe housing 402, and the packaging 428C may be sealed to both a first end 404 of the syringe housing 402 and a neck of the second port 416B. The second port 416 may include a protective cap 428 coupled to an end of the second port 416. In examples, the port may be coupled to a protective cap 438 and/or disinfecting cap (not shown). In examples, a disinfecting cap (not shown) may be separately sealed and coupled to an end of the protective cap 428. A second packaging 440A may surround the piston 408 and may be sealed to packaging 428C, the first end 404 of the syringe housing, and/or the syringe flanges 105.

In examples, the adapter may include a second push-push valve. For example, a second push-push valve may be positioned on a neck of the second port and may help prevent the two mixed medicants from collecting within the neck of the second port 416B and/or spilling out from a second port 416A before the medicant is administered.

FIG. 4D is a perspective view of an alternative mixing and/or administration system 400D including a second packaging 440B surrounding the port 416B. The second packaging 440B may include a tear strip 442 configured to provide easy access to the port. The second packaging 440B may be sealed to at least a portion of packaging 428D, port 416B, and/or the protective cap 438.

FIG. 4E is a perspective view of an alternative mixing and/or administration system 400E having a packaging 428E sealed to a portion of the syringe housing in accordance with an example of the present disclosure. In the particular example, the packaging 428E surrounds a portion of the mixing and/or administration system 400E including the vial 426, the extension housing 422, the push-push valve 420, the adapter 412, the second port 416A, and the packaging may be sealed to a portion of the Luer-lock fitting 410 associated with the syringe housing 402. In examples, the packaging 428E may be sealed to a portion of the syringe housing 402.

FIG. 4F is a perspective view of an alternative mixing and/or administration system 400F including a second packaging 428F sealed to the portion of the syringe housing. The second packaging 428F surrounds the syringe housing 402 and the piston 408 and may be sealed to a portion of the syringe housing and/or piston 408 in order to limit or reduce movement of the syringe housing and piston within the packaging during the mixing process. That is, a user may more easily manipulate contents within the second packaging without exposing the medicant to the environment and without exposing the user administering the medicant to the content of the packaging during the mixing/administration process.

FIG. 4G is a perspective view of another alternative mixing and/or administration system 400G including a syringe device 444 in a first packaging 446A, an IV bag 448 in a second packaging 446B, and IV tubing 450 in a third packaging 446C.

The intravenous (IV) bag 448 may be a flexible container used to administer fluids, medications, nutrients, etc. into a bloodstream of a patient, animal, etc. In some examples, the IV bag 448 may comprise of a flexible material (e.g., plastic, polyvinyl chloride (PVC), polyolefins, ethylene vinyl acetate, etc.). The IV bag may comprise various sizes (e.g., 100 mL to 1000 mL). Contents of the IV bag may include, for example, saline solution, electrolyte solution, glucose, blood, and various other liquids and solutions. The IV bag 448 may be surrounded by a second packaging 446B. The second packaging 446B may be sealed to or coupled to (in whole or in part) to a portion of the first packaging 446A surrounding a syringe device 444, a third packaging 446C surrounding the IV tubing 450, and/or a fourth packaging 446D surrounding a port associated with the syringe device 444. In some examples, the second packaging 446B may sealed to or coupled to a portion of a fourth packaging 446D surrounding a port associated with the syringe device 444, as shown in FIG. 4G. This is merely an example and the second packaging 446B surrounding the IV bag 448 may be coupled to a portion of the first packaging 446A (e.g., a side opposite the fourth packaging surrounding the port, proximate the tear open-pouch including the disinfecting cap(s), a side opposite the tear open-pouch, etc.). In examples, the second packaging 446B may comprise a tear strip 452 that provides easy access to the IV bag 448.

The intravenous (IV) tubing 450 may comprise of a flexible, transparent material and be configured to deliver fluids, medications, blood products, etc. from an IV bag or other container to a patient's bloodstream. In examples, the IV tubing 450 may include connectors on one or both ends of the IV tubing that enables the IV tubing to be connected to an IV bag or container, an IV catheter, needle, etc. In examples, a third packaging 446C may surround the IV tubing 450. In at least one example, the IV tubing may be included in a packaging associated with the IV bag. In examples, the third packaging 446C surrounding the IV tubing 450 may be sealed to a portion of the first packaging 446A surrounding the syringe device 444. In examples, the third packaging 446C may comprise a tear strip 454 that provides easy access to the IV tubing 450.

FIG. 4H is a perspective view of another alternative mixing and/or administration system 400H including a syringe in a first packaging 446A, an IV bag in a second packaging 446B, and IV tubing in a third packaging 446D, in accordance with an example of the present disclosure. In the particular example, the first packaging 446A is sealed to or coupled to a portion of the second packaging 446B via a first connection point 456A and is sealed to or coupled to the third packaging 446C via a second connection point 456B. The first connection point and the second connection point may comprise a strip of plastic or other material that is heat sealed or bonded (e.g., via adhesive) to a portion of the first packing, the second packaging, and the third packaging. The first connection point and the second connection point may enable the first packaging 446A to fold over the first and second packaging. Though two connection points are shown in FIG. 4H, any number of connection points may be utilized. Additionally, though the first packaging 446A, the second packaging 446B, and the third packaging 446C are shown in a particular configuration, it is contemplated that other combinations are possible. For example, the first packaging may be coupled to a side of the second packaging and not the third packaging. Alternatively, the first packaging may be coupled to a side of the third packaging and not the second packaging. In at least one example, the first packaging 446A, the second packaging 446B, and the third packaging 446C are not sealed or coupled to one another, but rather are disposed in a fourth packaging (not shown) that is sealed from the environment.

Features from any of the above-mentioned examples may be used in combination with one another in accordance with the general principles described herein. These and other examples, features, and advantages will be more fully understood upon reading the following detailed description in conjunction with the accompanying drawings and claims.

### CONCLUSION

Although the discussion above sets forth example implementations of the described techniques and structural features, other architectures may be used to implement the described functionality and are intended to be within the scope of this disclosure. Furthermore, although the subject matter has been described in language specific to structural features and/or methodological acts, it is to be understood that the subject matter defined in the appended claims is not necessarily limited to the specific features or acts described. Rather, the specific features and acts are disclosed as example forms of implementing the claims. For example, the structural features and/or methodological acts may be rearranged and/or combined with each other and/or other structural features and/or methodological acts. In various examples, one or more of the structural features and/or methodological acts may be omitted.

1. A mixing and administration system comprising:
a syringe housing having a first end and a second end opposite the first end, the first end associated with a connector;
a piston coupled to the second end of the syringe housing, the piston including a first end portion and a second end portion opposite the first end;
a push-push valve located proximate the second end portion of the piston;
an extension housing coupled to the second end portion of the piston, the extension housing having an open end;
a vial coupled to the open end of the extension housing; and
a packaging surrounding at least a portion of the mixing and administration system.

2. The mixing and administration system of claim 1, further comprising:
a sealed pouch coupled to the packaging and including a disinfecting cap.

3. The mixing and administration system of claim 2, wherein the sealed pouch includes a tear strip configured to provide access to the disinfecting cap without exposing the vial to an external environment outside the packaging.

4. The mixing and administration system of claim 1, wherein the packaging surrounds the extension housing, the vial, the push-push valve, and is coupled to the second end portion of the piston proximate the push-push valve.

5. The mixing and administration system of claim 1, wherein the packaging surrounds the extension housing, the vial, the piston, and is coupled to the second end of the syringe housing.

6. The mixing and administration system of claim 1, wherein the packaging surrounds the extension housing, the vial, the piston, the syringe housing, and is coupled to the first end of the syringe housing.

7. The mixing and administration system of claim 1, wherein the syringe housing and the first end of the piston are exposed to an environment outside the packaging.

8. The mixing and administration system of claim 1, wherein the first end of the syringe housing associated with the connector is exposed to an environment outside the packaging.

9. The mixing and administration system of claim 1, further comprising:
a Luer connector coupled to the first end of the syringe housing;
a protective cap coupled to the Luer connector; and
a disinfecting cap coupled to the protective cap.

10. The mixing and administration system of claim 1, wherein a portion of the packaging is coupled to a section of the syringe housing such that movement of the syringe within the packaging is reduced.

11. The mixing and administration system of claim 1, wherein the connector associated with the syringe housing is coupled to a disinfecting cap.

12. The mixing and administration system of claim 1, further comprising:
a Luer connector sealed to a portion of the packaging located proximate the connector of the syringe housing.

13. The mixing and administration system of claim 1, wherein the push-push valve is partially sealed to the packaging.

14. The mixing and administration system of claim 1, wherein activating the push-push valve causes a channel to open between the syringe housing and the extension housing including the vial.

15. The mixing and administration system of claim 1, wherein the piston includes a raised ridge positioned proximate the second end of the piston, the raised ridge configured to prevent the syringe housing from abutting the push-push valve.

16. A mixing and administration system comprising:
a syringe housing having a first end and a second end opposite the first end;
a piston coupled to the first end of the syringe housing;
a packaging surrounding a first portion of the mixing and administration system such that a second portion of the mixing and administration system different than the first portion is exposed to an external environment to the mixing and administration system; and
a sealed pouch coupled to the packaging, the sealed pouch including at least one cleaning device.

17. The mixing and administration system of claim 16, wherein the at least one cleaning device includes a disinfecting cap, a sponge applicator, an alcohol wipe, or a swab.

18. The mixing and administration system of claim 16, wherein the sealed pouch includes a tear strip configured to provide access to the cleaning device without exposing the first portion of the mixing and administration system to the external environment outside the packaging.

19. (canceled)

20. (canceled)

22. The mixing and administration system of claim 16, wherein a part of the first portion of the packaging is coupled to a section of the syringe housing such that movement of the syringe within the packaging is reduced.

23. The mixing and administration system of claim 16, wherein a part of the first portion of the packaging is coupled to a section of the piston such that movement of the syringe within the packaging is reduced.
